(19) United States
(12) Patent Application Publication
(10) Pub. No.: US 2006/0099677 A1
(43) Pub. Date: May 11, 2006

(54) DNA/RNA transduction technology and its clinical and basic applications

(76) Inventors: Sang-Kyu Lee, Seoul (KR); Seung-Kyu Lee, Daejeon (KR); Wook-Jin Chae, Seoul (KR); Je-Min Choi, Seoul (KR); Jung-Jin Yang, Seoul (KR)

Correspondence Address: Neil A DuChez;Renner Otton Boisselle & Sklar, 1621 Euclid Avenue, 19th Floor, Cleveland, OH, 44115, US

(21) Appl. No.: 10/534,433

(22) PCT Filed: Nov. 12, 2003
(86) PCT No.: PCT/KR03/02425

§ 371 Date: Nov. 21, 2005

(30) Foreign Application Priority Data
Nov. 12, 2002 (KR) 10-2002-0070106

(51) Int. Cl. C12P 21/06; C12N 15/63

(52) U.S. Cl. 435/69.1; 435/455

(57) ABSTRACT

This invention relates to a method for in vivo and in vitro transferring efficiently DNA/RNA coding materials regulating bio-function to cytoplasm or nucleus in eukaryotic or prokaryotic cells using PTD (Protein Transduction Domain) and DNA/RNA binding factor. Particularly, this invention provides a method for in vivo transferring the materials to cells through various routes comprising intramuscular, intraperitoneal, intravein, oral, nasal, subcutaneous, intradermal, mucosal, inhalation. Accordingly, the method of this invention can be used for technology to transfer DNA/RNA to various cell types and express them in the cells transiently or permanently in medicinal applications such as DNA/RNA vaccine and gene therapy, as well as basic applications.

### TECHNICAL FIELD

This invention relates to a method for effective transferring of DNA/RNA encoding biological regulator in vivo and in vitro into eukaryotic or prokaryotic cytoplasm or nucleus, using PTD (Protein Transduction Domain) and DNA/RNA binding factor.

### BACKGROUND ART

This invention relates to a system for effective transduction of biological regulator in vivo and in vitro into eukaryotic or prokaryotic cytoplasm or nucleus.

Generally, a living cell is impermeable to macromolecules such as proteins or nucleic acids. The fact that some small substances can pass through the plasmic membrane of the living cell in low rate, but macromolecules cannot permeate the cell membrane, is a limiting factor of medical treatment, prevention, and diagnosis using macromolecules including proteins and nucleic acids. Meanwhile, because most of substances made for medical treatment, prevention, and diagnosis must be delivered with their effective amount for medical treatment, prevention, and diagnosis, various methods have been developed to deliver these substances into the cell by making them react to the exterior of the cell or surface of the target cell. Thus, there are methods to deliver in vitro macromolecules into the cell, for example, electroporation, membrane fusion using liposomes, high-concentration projection using particular projectors coated with DNA, cultivation using calcium-phosphorous-DNA precipitate, DEAE-dextran transfection, infection of modified viral nucleic acid, direct injection to a single cell. However, typically, these methods can only deliver macromolecules to a portion of the target cells, and can cause side effects to many other cells. Also, there are methods to deliver in vivo macromolecules into the cell, for example, scrape loading, calcium-phosphate precipitation, method using liposomes but a controversial point is that the usage of these methods has in vivo limitations.

Therefore a general way to deliver in vivo and in vitro biologically active macromolecules into the cell without damaging the cell was needed [Reference: L. A. Sternson, Ann. N.Y. Acad. Sci., 57, 19-21 (1987)]. For example, there is chemical addition of lipid peptide [Reference: P. Hoffmann et al. Immunobiol., 177, 158-170(1988)] or method using basic polymers such as polylysine or polyarginine [Reference: W-C. Chen et al., Proc. Natl. Acad. Sci., USA, 75, 1872-1876(1978)], but these methods are not verified yet. Folic acid used as transporters [Reference: C. P. Leamon and Low, Proc. Natl. Acd. Sci., USA, 88, 5572-5576(1991)] was reported that it moves into the cell as folic acid-salt complex but it is not confirmed whether it can be delivered into the cytoplasm. Likewise, Pseudomonas Exotoxin is also used as a kind of transporter [Reference: T. I. Prior et al., Cell, 64, 1017-1023(1991)]. Nevertheless, the effect by the delivery of the biologically active substance which has to be delivered into the cell and its possibility of application is not clear. Consequently, a way to deliver biologically active substances into the cell more safely and more effectively is required continually.

As a result of study for the requirement, PTDs (Protein Transduction Domain) were reported, and among various PTDs, the transcription factor, Tat, of Human Immunodeficiency Virus-1, HIV-1 is studied the most. This protein can pass through the cell membrane more effectively when it is organized by part of the amino acids distributed through 47 to 57 (YGRKKRRQRRR) where positively charged amino acids are distributed than when it is a complete form consisting of 87 amino acids [Reference: Fawell S. et al. Proc. Natl. Acad. Sci. USA 91, 664-668(1994)]. Like this, amino acids 267 to 300 of VP22 protein of Herpes Simplex Virus type 1 [Reference: Elliott G. et al. Cell, 88,223-233(1997)], amino acids 84 to 92 of UL-56 protein of HSV-2 (GeneBank code: D1047[gi:221784]), and amino acids 339 to 355 of ANTP(Antennapedia) protein of Drosophila [Reference: Schwarze S. R. et al. Trends Pharmacol Sci. 21, 45-48(2000)] are examples of other PTDs, and artificial peptides consisting of positively charged amino acids also showed effects [Reference: Laus R. et al. Nature Biotechnol. 18, 1269-1272(2000)].

We used fusion proteins obtained by fusing DNA/RNA binding factor or the binding domains of DNA/RNA to PTD, and ameliorated the delivery of regulatory molecules such as DNA/RNA into the cell. Moreover, the invention was completed by the discovery that regulatory molecules can be delivered to specific cells, tissues, and organs specifically or by the induction with specific stimulus.

### DISCLOSURE OF INVENTION

The purpose of this invention is to in vivo and in vitro bind a fusion protein of PTD with a DNA/RNA encoding regulatory proteins containing selectable DNA Binding Sequence, DBS, and DNA/RNA binding factor which can be combine with DBS, or homologous or heterologous one or more binding protein comprising DNA Binding Domain, DBD, thereafter, to transfer a DNA/RNA encoding a biological regulatory ex vivo into strains or in vivo into each organ through routes including intramuscular, intraperitonea, intravein, oral, nasal, subcutaneous, intradermal, mucosal or inhalation by PTD, and to express the proteins. Especially, when DNA/RNA encoding the biological regulator comprises a promoter which can regulate the expression of the DNA/RNA at a specific organ, tissue or cell, the biological regulator can be expressed at a specific target site.

Another purpose of this invention is to transduce one or more biological regulators selected from the group consisting of protein, DNA/RNA, fat, carbohydrates, or chemicals in vitro or in vivo into eukaryotic or prokaryotic cytoplasm or nucleus.

Furthermore, the purpose of this invention is to provide a novel method for gene therapy and DNA/RNA vaccine using the method of the present invention, and a method of transducing the target DNA/RNA fragments into various kinds of prokaryotic and eukaryotic cells, and a method for permanently or transiently expressing the proteins with the target DNA/RNA.

### BRIEF DESCRIPTIONS OF DRAWINGS

FIG. 1a to FIG. 1c are structures of recombinant expression vectors of this invention.

FIG. 2a and FIG. 2b are photographs of agarose gels after electrophoresis of the expression vectors of FIG. 1 digested with restriction enzymes.

FIG. 3 is a result of coomassie blue staining of the purified fusion protein expressed from the expression vectors.

FIG. 4 indicates the detection of CD8-z, Lck protein delivered into Jurkat T cells by Sim2-Gal4, Mph1-Gal4, or R7-Gal4 through western blot analysis using mAb of CD8 and Lck.

FIG. 5 indicates the detection of CD8-z, Lck protein delivered into Hela cells by Sim2-Gal4, Mph1-Gal4, or R7-Gal4 through western blot analysis using mAb of CD8 and Lck.

FIG. 6a to FIG. 6d indicate the detection of CD8-z and Lck expressed in the heart (FIG. 6a), liver (FIG. 6b), kidney (FIG. 6c), and spleen (FIG. 6d) of mouse using their mAb, after injecting pCD8-z-GBS or pLck-GBS with Sim2-Gal4, Mph1-Gal4, Tat-Gal4, or R7-Gal4 through I.P.

FIG. 7a to FIG. 7c indicate specific expressions of each protein after injecting pL-CD8-z-GBS or pL-Lck-GBS with Sim2-Gal4, Mph1-Gal4, Tat-Gal4, or R7-Gal4 through I.P.

FIG. 8a to FIG. 8c indicate specific expressions of each protein after injecting pL-CD8-z-GBS or pL-Lck-GBS with Sim2-Gal4, Mph1-Gal4, Tat-Gal4, or R7-Gal4 through I.P.

In order to accomplish the above purpose, this invention provides a protein tranducing recombinant expression vector, comprising a fusion protein of PTD (Protein Transduction Domain) with one or more homologous or heterologous binding proteins having DNA/RNA Binding Domain (DBD) or DNA/RNA binding factor that is able to combine with specific DNA/RNA binding sequences, a DNA encoding the binding each proteins, and a DNA encoding PTD, and expression regulatory sequences operatively bound to the vector.

Moreover, the invention provides a recombinant expression vector, comprising a DNA/RNA encoding a biological regulator protein which contains DNA/RNA binding sequence at 3′ or 5′, wherein the sequence binds specifically to the DNA/RNA binding factor or DNA/RNA binding domain, and a selective promoter as expression regulatory sequence operatively bound to the vector in cells, tissues or organs expressing the proeins.

In addition, this invention provides a DNA structure including one or more biological regulators selected from the group consisting of protein, DNA/RNA, fats, carbohydrates and chemicals, wherein DNA/RNA binding sequences that specifically bind above DNA/RNA binding factor or DNA/RNA binding domain by chemical or physical non-covalent or covalent bond.

The invention also provides a binding complex to deliver a biological regulator into the cytoplasm or the nucleus, wherein, the binding complex comprises a fusion protein of PTD with one or more homologous or heterologous binding protein that has DNA/RNA binding factors or DNA/RNA binding domains; and one or more biological regulators selected from the group consisting of protein, DNA/RNA, fats, carbohydrates and chemicals are combined by chemical or physical non-covalent or covalent bond.

Moreover, this invention provides a binding complex to deliver DNA into the cytoplasm or the nucleus, wherein the binding complex comprises a fusion protein of PTD with one or more homologous or heterologous binding protein that contains DNA/RNA binding factors or DNA/RNA binding domains; and DNA/RNA binding sequences that specifically combine with the above DNA/RNA binding factors or DNA/RNA binding domains, DNA encoding biological regulator and a recombination expression vector that operatively binds the expression regulatory sequences.

In addition, using the fusion protein of PTD with the protein having DNA/RNA binding factors or DNA/RNA binding domains (DBD), this invention provides a method for expressing DNA/RNA that encodes biological regulatory containing DBS, capable of selectively binding above binding factors or DBD, by delivering it into prokaryotic or eukaryotic cytoplasm or nucleus after contacting with prokaryotic or eukaryotic cells through various routes including intramuscular, intraperitoneal, intravein, oral, nasal, subcutaneous, intradermal, mucosal or inhalation ex vivo or in vivo.

This invention provides a method for delivering a biological regulator to eukaryotic or prokaryotic cytoplasm or nucleus, comprising steps i) preparing a transducing recombinant expression vector that operatively binds to the expression regulatory sequences as well as DNA which encodes PTD and one or more homologous or heterologous binding protein that has DNA/RNA binding factor or DNA/RNA binding domain; ii) obtaining a fusion protein by expressing the recombinant expression vector of step i) in a host cell; iii) obtaining a binding complex by coupling the fusion protein of step ii) and one or more biological regulators selected from the group consisting of protein, DNA/RNA, fats, carbohydrates and chemicals, through chemical or physical non-covalent or covalent bonds; iv) mixed-culturing the binding complex of step iii) and cell culture through routes including intramuscular, intraperitoneal, intravein, oral, nasal, subcutaneous, intradermal, mucosal or inhalation ex vivo or in vivo.

This invention provides a method for delivering biological regulators to eukaryotic or prokaryotic cytoplasm or nucleus, comprising steps: i) preparing a transducing recombinant expression vector that operatively binds to the expression regulatory sequences as well as DNA which encodes PTD and one or more homologous or heterologous binding protein that has DNA/RNA binding factor or DNA/RNA binding domain; ii) obtaining a fusion proteins by expressing the recombinant expression vector of step i) in a host cell; iii) preparing a recombinant expression vector which comprises a DNA encoding a biological regulator, DNA/RNA sequences to be bound specifically to the DNA/RNA binding factor or the DNA/RNA binding domain, and an expression vector operatively bound to the vector; iv) obtaining a binding complex by combining the fusion protein obtained from step ii) and the recombination expression vector of step iii); v) mixed-culturing the binding complex of step iv) and cell culture through routes including intramuscular, intraperitoneal, intravein, oral, nasal, subcutaneous, intradermal, mucosal or inhalation ex vivo or in vivo.

Using the fusion proteins of PTD with the binding proteins having DNA/RNA protein factors or DNA/RNA binding domains(DBD), the invention also provides a method of delivering biological regulators, linked with DBS that selectively combines with the above binding factors or DBD by chemical or physical non-covalent or covalent bonds, into prokaryotic or eukaryotic cytoplasm or nucleus after contacting with prokaryotic or eukaryotic cells through various routes including intramuscular, intraperitoneal, intravein, oral, nasal, subcutaneous, intradermal, mucosal or inhalation ex vivo or in vivo.

In this invention, “PTD” refers to a transportable peptide that delivers particular proteins, either directly linked by chemical or physical covalent or non-covalent bonds or indirectly linked using other medium, into eukaryotic or prokaryotic cytoplasm or nucleus. They include, but not limited to, Sim-2 [bibliography: Chrast R. et al. Genome Res. 7, 615-624 (1997)], Mph1 [bibliography: M. J Alkema et al., Genes Dev. 11(2), 226-240(1997)], Tat [bibliography: Fawell S. et al. Proc. Natl. Acad. Sci. USA 91, 664-668(1994)], R7 (Cell Gate, U.S.A.), SM5 (Dr. Quin, Vanderbilt University), VP22 [bibliography: Elliott G. et al. Cell, 88,223-233(1997)], ANTP [bibliography: Le Roux I. et al, Proc. Natl. Acad. Sci. USA 90, 9120-9124(1993)], Pep-1 and Pep-2[bibliography: May C. Morris et al, Nature Biotechnology, 19, 1173-1175(2001)].

“DNA/RNA binding factor” or “DNA/RNA binding domain (DBD)” refers to the whole protein or part of the protein which can bind to specific DNA/RNA sequence, and includes transcriptional factor or viral protein.

“Binding protein” refers to a DNA/RNA binding factor or homologous or heterologous one or more the protein, which has DNA/RNA binding domain.

“Selective promoter” is a promoter which can express a gene encoding a protein in specific tissue, cell or organ. For example, there are T-cell-specific Lck, CD2 promoter, and pancreas-specific insulin promoter. Also, the promoter may be an inducible promoter.

The invention also provides a transducing recombinant expression vector which includes DNA encoding PTD and DNA encoding a protein having DBD.

The transducing recombinant expression vector may comprise a tag sequence which makes it easy to purify the fusion protein—for example, continuous histidine codon, hemaglutinine codon, Myc codon, and maltose binding protein codon. Additionally, the vector can include, but not limited to, expression controlling sequence and restriction sequence to be cut by specific enzymes such as enterokinase to remove unfavorable part from the fusion protein, for example, factor X, Thrombin and marker or reporter gene sequence to confirm the delivery.

As shown in following Examples, transducing recombinant expression vector pPTD-GAL4, for example, pSim2-Gal4, pMph1-Gal4, pTat-Gal4 or pR7-Gal4, includes PTD: Sim-2, Mph-1, Tat or R7 coding DNA. It also includes six-His codon, for purification of protein expressed from host cell, Asp-Asp-Asp-Asp-Lys sequence restricted specifically by enterokinase, and DNA encoding Gal4 DNA binding factor that can bind to Gal4 binding sequence specifically.

The vector pPTD-Gal4 of this invention can be prepared by PCR (polymerase chain reaction) simply using pTrcHisB (Invitrogen) as template. In addition, according to this invention, various kinds of the recombinant expression vector can be prepared by cutting out Gal4 gene (Invitrogen) from the vector using an appropriate restriction enzyme and replacing it with other DNA encoding whole or part of DNA binding factor which can bind to specific DNA sequence. In the following examples, Gal4 is used as DNA Binding Factor. Gal4 is originally a transcription factor which is widely used in eukaryotes, prokaryotes, and viruses. Gal4 in this invention can form binding protein fused chemically or physically with monoclonal antibody which specifically binds to certain kinds of receptors or ligands expressed on a specific cell to enhance the specificity. The substances to be fused with Gal4 are not only protein fragments but also fats, carbohydrates, or their complexes. Gal4 fusion protein complex of this invention includes, but not limited to, DNA, RNA, carbohydrates, lipids bound with transducing peptide chemically or physically.

In order to obtain a protein fused with transducing peptide as target protein using transducing recombinant expression vector, we transformed appropriate host cells such as E. coli with recombinant expression vector and obtained fusion protein expressed from those transformant, and then separated target protein according to ordinary protocols such as poly Histidine and Ni²⁺-NTA methods, and purified it if necessary.

In addition, this invention provides a method for transducing, comprising steps i) obtaining binding complexes by fusion with target biological regulators after activating transducing peptides or their derivatives or fusion protein of transducing peptide with binding protein using binding inducer; ii) delivering target biological regulators into cells by mixed-culturing the binding complexes with the cell culture. Furthermore, NLS (nuclear localization sequence) can bind to PTD of fusion protein. The aforementioned fused derivatives include reagents which combine PTD (protein transducing domain) or fused protein of PTD with target protein, with target biological regulators (e.g. DNA, RNA, carbohydrates, fats, protein or chemicals) by physical or chemical means. Examples of such reagents would be BMOE (Pierce Cat. No 22323), DSP (Pierce Cat. No 22585), etc.

Moreover, on delivering the functional regulator into the cell, if fusion proteins of transducing peptide with binding protein are delivered to specific cells, tissues or organs, the binding protein may be receptors or ligands which are expressed in specific cells, tissues or organs, or mAb and its derivatives which can interact with the ligands.

Meanwhile, the biological regulator can be promoter or enhancer in itself that can express a gene in specific species, tissues, organs, or cells.

Due to extremely small size, the transducing peptides in this invention are able to minimize the possible occurrence of biological interference with other biologically active substances.

This invention will be described in more detail by the example given below. However, it is intended that the examples are considered exemplary only and the scope of the invention is not limited.

### EXAMPLES

### Example 1

### Preparation of Recombinant Expression Vector

Preparation of Transducing Recombinant Expression Vector for Fusion Protein of Transducing Peptide with Binding Protein having DBD (pSim2-Gal4, pMph1-Gal4. pTat-Gal4, pR7-Gal4, pCD8-z-GBS).

We used Sim-2 gene (alanine at 558˜arginine at 566 from N terminus), Mph-1 gene (tyrosine at 858˜arginine at 868 from N terminus), Tat gene of HIV (tyrosine at 47˜arginine at 57 from N terminus), or base sequence encoding peptides consisting of 7 arginine amino acids as protein transducing peptides. We used Gal4 (Invitrogen) as binding proteins with DBD. In order to combine the above protein transducing peptides with base sequence encoding Gal4 to be bound to Gal4-binding sequence (GBS), SEQ ID NO: 1˜4 corresponding to primers for Sim-2, Mph-1, Tat and 7 arginines, primer of SEQ ID NO: 5 corresponding to 3′ end of Gal4 to prepare the DNA structures and BamH I site for cloning was synthesized. And then, PCR was carried out with pfu turbo DNA polymerase (Stratagen) using the vector containing whole gene of the Gal4 protein (Clontech) as template.

In this example 1, SEQ ID NO: 1 is 5′ primer for pSim2-gal4, SEQ ID NO: 2 is 5′ primer for pMph1-Gal4, SEQ ID NO: 3 is 5′ primer for pTat-Gal4, SEQ ID NO: 4 is 5′ primer for pR7-Gal4, and SEQ ID NO: 5 is 3′ primer for pSim2-Gal4, pMph1-Gal4, pR7-Gal4, and pTat-Gal4.

Preparation of the Recombinant Vector Which Contains DNA Binding Sequence (DBS) and Encodes Biological Regulators (pLck-GBS pINS-GBS pL-CD8-z-GBS. pL-LCK-GBS pL-INS-GBS).

We prepared primers of SEQ ID NO: 6 and 7, wherein the sequences, expression vectors pCDNA-Lck or pCDNA-INS comprise a gene encoding Lck or insulin. Also, the sequences comprise Gal4 binding sequence (GBS) specifically binding to DNA binding sequence, Gal4, in restriction enzyme sites and Stu I at 5′ and 3′. And then, we carried out PCR using pGAD as template. The resulting reaction matter from PCR was purified with the PCR purification kit (Qiagen) and digested with Bgl II and BamHI restriction enzymes for 48 hours. Then it was purified separately by 1% agarose gel elecrophoresis, and stained with ethidium bromide.

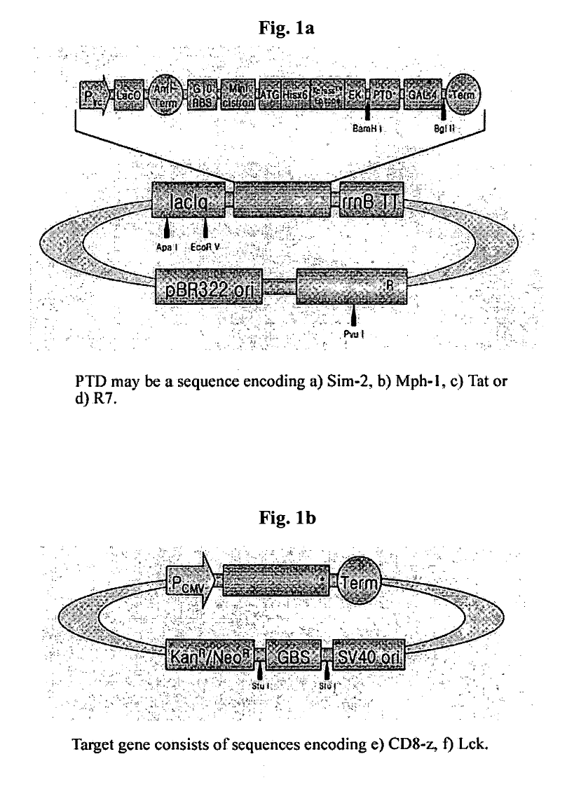

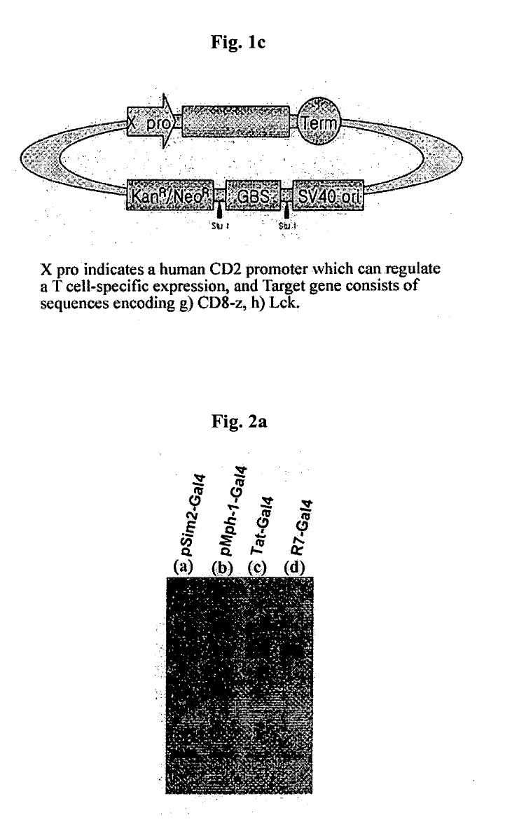

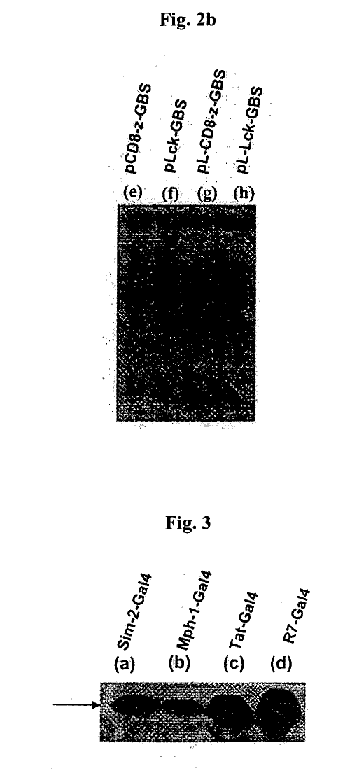

Also, each DNA of Lck-GBS, INS-GBS, and CD8-z-GBS were separated from pLck-GBS, pINS-GBS, and pCD8-z-GBS with restriction enzymes and cloned with the expression vector pLck-Luc which is selectively expressed in T cell. The recombinant expression vector prepared through cloning in the above method was named pSim2-Gal4(a), pMph1-Gal4(b), pTat-Gal4(c), pR7-Gal4(d), pCD8-z-GBS(e), pLck-GBS(f), pINS-GBS(g), pL-CD8-z-GBS(h), pL-Lck-GBS(i), and pL-INS-GBS(j), and their structures are shown in FIG. 1a-1c.

SEQ ID NO: 6 is base sequence of 5′ primer for preparation of Gal4 Binding Sequence (GBS), and SEQ ID NO: 7 is base sequence of 3′ primer for preparation of Gal4 Binding Sequence.

### Example 2

### Preparation of E. coli Transformant and Expression and Purification of Fusion Protein

E. coli DH5 (ATCC No. 53863) was transformed with the expression vectors, pSim2-Gal4(a), pMph1-Gal4(b), pTat-Gal4(c), pR7-Gal4(d) prepared in Example 1 using heat shock transformation. Then, 2 ml of the transformant was inoculated to 100 ml of LB medium and pre-cultured with agitation at 37° C. for 12 hours. Next, after the resulting culture was inoculated to 1000 ml of LB medium and cultured at 37° C. for 4 hours, the expression of 1 ac operon was induced by adding 1 mM of IPTG (Isopropyl-D-thiogalactopyranoside, GibcoBRL cat. # 15529-019). Then, it was cultured for another 8 hours to induce the expression of fusion protein.

The above culture was centrifuged at 6,000 rpm at 4° C. for 20 minutes to remove the supernatant. The remaining pellets were dissolved in 10 ml of buffer solution 1 (50 mM NaH₂PO₄, 300 mM NaCl, 10 mM imidazole, pH 8.0) containing 1 mg/Me of lysozyme (Sigma, cat.# L-7651) and placed on ice for 30 minutes. Then, the solution was treated with supersonic waves with the intensity of 300 W for 10 seconds using a supersonic homogenizer (Heat System, Ultrasonic Processor XL), and then chilled for 10 seconds. This was repeated so that the total cumulated time for supersonic wave exposure was 3 minutes. The effluent was centrifuged at 12,000 rpm at 4° C. for 20 minutes to remove the fragments of the debris and separate only the pure effluent.

2.5 Me of 50% Ni²⁺-NTA agarose slurry (Qiagen, cat# 30230) was added to the effluent and mixed for 1 hour at 200 rpm at 4° C. to combine the fusion protein with Ni²⁺-NTA agarose. This mixture was put through a 0.8×4 cm chromatography column (BioRad, cat. # 731-1550). The fusion protein was washed twice with 4 ml of buffer solution 2 (50 mM NaH₂PO₄, 300 mM NaCl, 20 mM imidazole, pH 8.0) and then fractioned 4 times using 0.5 Me of buffer solution 3 (50 mM NaH₂PO₄, 300 mM NaCl, 250 mM imidazole, pH 8.0). FIG. 3 shows the result of a coomassie blue staining after SDS-PAGE was carried out. Lane 1 of FIG. 3 shows the standard molecular weight of protein and it is Sim2-Gal4(a), Mph1-Gal4(b), Tat-Gal4(c), and R7-Gal4(d), respectively.

### Example 3

### Delivery and Expression of the DNA into Jurkat T cell by Sim2-Gal4, Mph1-Gal4, Tat-Gal4, or R7-Gal4 (in vivo)

After combining the fusion protein Sim2-Gal4, Mph1-Gal4, Tat-Gal4, and R7-Gal4 resulted from Example 2 with linear DNA structure pCD8-z-GBS, pLck-GBS, pINS-GBS at 37° C., 1 ml of Jurkat cells (ATCC No. TIB-152) were added to 35 mm Petri dish and reacted at 37° C. for 30 minutes. The reaction was terminated and collected cells, and the cells were reacted in 100 ml of elution buffer solution [0.2% triton X-100, 150 mM NaCl, 10 mM Tris-HCl, 400 M EDTA, 1 mM Na₃VO₄, 10 mM NaF, 1 mM PMSF, 10 g aprotinin, 10 g leupeptin] at 4° C. for 30 minutes and then centrifuged at 14,000 rpm for 15 minutes to obtain the cell elution solution.

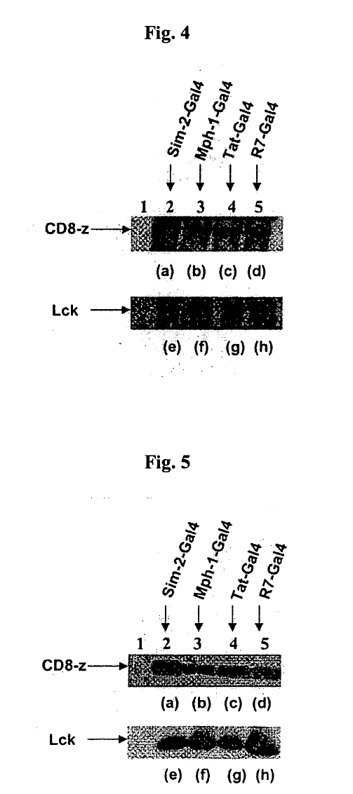

This cell elution solution was separated with SDS-PAGE gel, and the expressed protein was detected through Western Blot analysis using mAb (OKT8) for CD8, mAb for Lck, mAb for INS. The result is shown in FIG. 4 (the result of INS is not shown). The first lane shows the standard molecular weight of protein and it is delivery by Sim-2-Gal4: CD8-z (a), Lck (e); delivery by Mph1-Gal4: CD8-z (b), Lck (f); delivery by Tat-Gal4: CD8-z(c), Lck (g); and delivery by R7-Gal4: CD8-z (d), Lck (h), respectively.

### Example 4

### Delivery and Expression of DNA into Hela cell by Sim2-Gal4, Mph1-Gal4, Tat-Gal4, or R7-Gal4 (in vitro)

Using the same method as in Example 3, pCD8-z-GBS, pLck-GBS, and pINS-GBS combined with Sim2-Gal4, Mph1-Gal4, Tat-Gal4, and R7-Gal4 was delivered to the Hela cell. And CD8-z, Lck, and insulin (INS) expressed in the cell was detected using Western Blot analysis. The result except insulin, is shown in FIG. 5. Lane 1 shows the standard molecular weight of protein, and delivery by Sim2-Gal4: CD8-z (a), Lck (e); delivery by Mph1-Gal4: CD8-z (b), Lck (f); delivery by Tat-Gal: CD8-z(c), Lck (g); and delivery by R7-Gal4: CD8-z (d), Lck (h), respectively.

### Example 5

### Delivery and Expression of DNA by Sim2-Gal4, Mph1-Gal4, Tat-Gal4, or R7-Gal4 (in vivo)

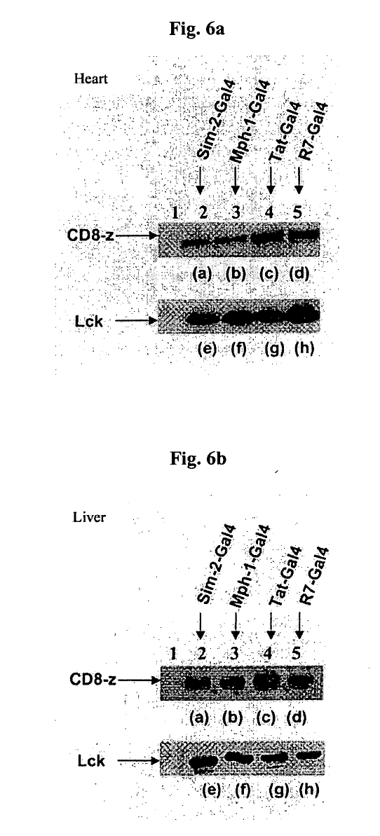

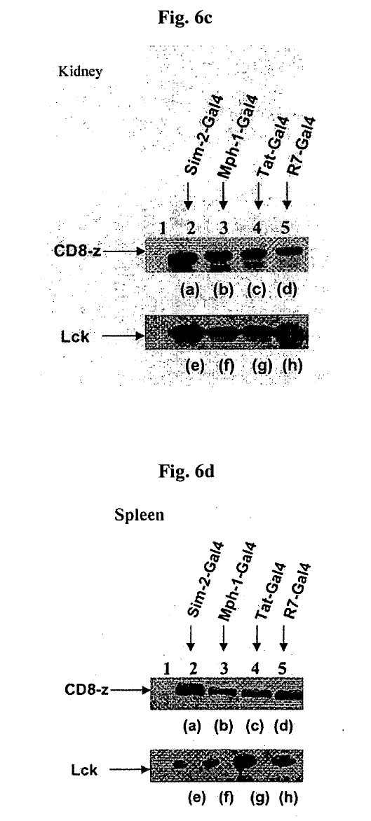

Fusion protein, Sim-Gal4, Mph1-Gal4, Tat-Gal4, or R7-Gal4 prepared in Example 4 and pCD8-z-GBS, pLck-GBS, pINS-GBS was fused to form protein complex. 0.5 mg/ml of this complex is injected by I.P. into C57B6 mouse. After 4 hours, several organs, heart, liver, and spleen, are extracted. Western blot analysis methods are used to detect CD8-z, Lck, and insulin expressed on the surface due to this protein complex. The results except insulin are shown in FIG. 6a to 6d.

i) Heart (FIG. 6a): The first lane is standard molecular weight of protein and it is delivered by Sim2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

ii) Liver ( FIG. 6b): The first lane is standard molecular weight of protein and it is delivered by Sim2-Gal4: CD8-z(a), Lck(e); delivery by Mph1-Gal4: CD8-z(b), Lck(f); delivery by Tat-Gal4: CD8-z(a), Lck(g); delivery by R7-Gal4: CD8-z(d), Lck(h), respectively.

iii) Kidney (FIG. 6c): The first lane is standard molecular weight of protein and it is delivered by Sim2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

iv) Spleen (FIG. 6d): The first lane is standard molecular weight of protein and it is delivered by Sim2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

### Example 6

### Cell-Specific Expression of Target DNA in vivo by Sim2-Gal4, Mph1-Gal4, Tat-Gal4, or R7-Gal4

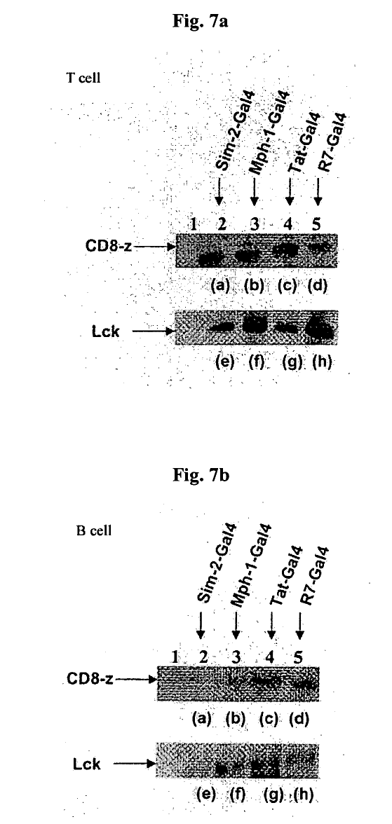

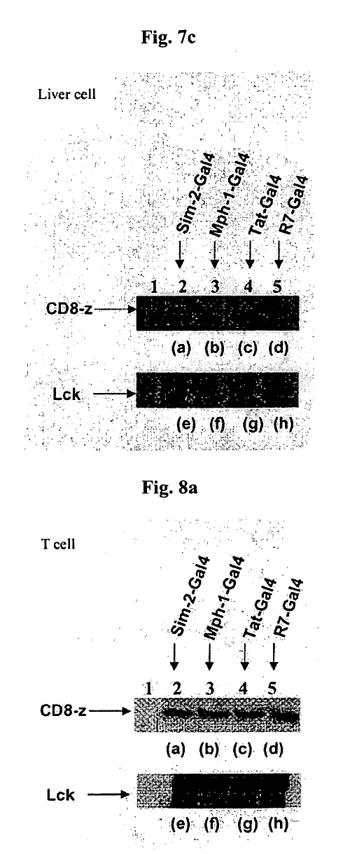

The plasmids prepared from Example 1, pL-CD8-z-GBS, pL-Lck-GBS, pL-INS-GBS, are linearized to fuse with Sim2-Gal4, Mph1-Gal4, Tat-Gal4, or R7-INS-Gal4 protein to make fusion protein complexes in 37□. 0.5 mg/ml of these complexes are injected by I.P. into C57B6 mouse. 4 hours later, liver, T-cells and B-cells are extracted, and western blot analysis methods are used to detect CD8-z, Lck, and insulin expressed on the surface due to this protein complex. The results of these experiments except insulin are shown in FIG. 7a to 7c.

i) T cell (FIG. 7a): The first lane is standard molecular weight of protein and it is delivered by Sim2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

ii) B cell (FIG. 7b): The first lane is standard molecular weight of protein and it is delivered by Sim2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

iii) Liver cell (FIG. 7c): The first lane is standard molecular weight of protein and it is delivered by Sim2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

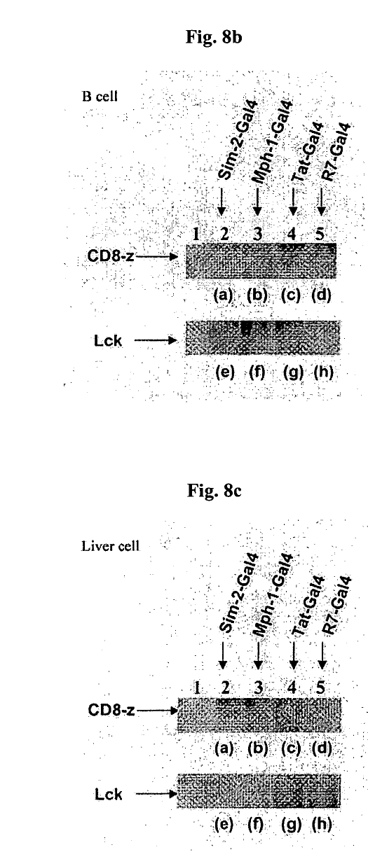

0.5 mg/ml of complex of the fusion proteins, Sim2-Gal4, Mph-Gal4, Tat-Gal4 or R7-Gal4 and pL-CD8-z-GBS, pL-Lck-GBS, or pL-INS-GBS DNA were injected epithermally to C57B6 mouse. Six hours later, liver, T-cells and B-cells are extracted, and western blot analysis methods are used to detect expressions of CD8-z, Lck, and insulin with their mAbs. The results of these experiments are shown in FIG. 8a to 8c.

i) T cell (FIG. 8a): The first lane is standard molecular weight of protein and it is delivered by Sim-2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

ii) B cell (FIG. 8b): The first lane is standard molecular weight of protein and it is delivered by Sim-2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

iii) Liver cell (FIG. 8c): The first lane is standard molecular weight of protein and it is delivered by Sim-2-Gal4: CD8-z (a), Lck (e); delivery by Mph1-Gal4: CD8-z (b), Lck (f); delivery by Tat-Gal4: CD8-z(c), Lck (g); delivery by R7-Gal4: CD8-z (d), Lck (h), respectively.

### INDUSTRIAL APPLICABILITIES

This invention relates to a technology that can deliver DNA effectively into cytoplasm or nucleus of eukaryotic or prokaryotic cell through various routes including intramuscular, intraperitoneal, intravein, oral, nasal, subcutaneous, intradermal, mucosal or inhalation, using DNA/RNA structure containing DNA/RNA binding factor which can be combined to PTD or specific DNA/RNA sequence, fusion protein which can be fused with binding protein that has DNA/RNA binding domain or DNA/RNA binding sequence which is specifically combined with biological regulator and DNA/RNA binding factor. This technology can be used to not only practical application for development of DNA/RNA vaccine and gene therapy, but also basic research that investigate function of protein which is expressed inside of cell continuously or temporarily by certain gene.

1. A binding complex for delivering DNA/RNA into cytoplasm or nucleus, comprising a fusion protein of PTD with one or more homologous or heterologous binding protein having DNA/RNA binding factor or DNA/RNA binding domain; a DNA/RNA binding sequence which is specifically bound to the DNA/RNA binding factor or DNA/RNA binding domain; and a DNA/RNA encoding biological regulator.

2. The binding complex according to claim 1, wherein NLS (Nuclear Localization Sequence) is additionally combined with PTD of fusion protein.

3. The binding complex according to claim 1, wherein PTD is selected from the group consisting of Mph-1, Sim-2, Tat, R7, VP22, ANTP, MTS<Pep-1, and Pep-2.

4. The binding complex according to claim 1, wherein the biological regulator is a promoter or enhancer that expresses a gene specifically in specific species, tissues, organs or cells.

5. The binding complex according to claim 4, wherein the promoter is an inducible promoter or enhancer.

6. The binding complex according to claim 1, wherein the complex is delivered into cytoplasm or nucleus through routes including intramuscular, intraperitoneal, intravein, oral, nasal, subcutaneous, intradermal, mucosal or inhalation.

7. A method for delivering a biological regulator into eukaryotic or prokaryotic cytoplasm or nucleus, comprising steps:
i) Preparing peptide transducing recombinant expression vector which comprises a DNA encoding PTD, a DNA encoding one or more homologous or heterologous binding protein having DNA/RNA binding factor or DNA/RNA binding domain, and expression regulatory sequence operatively bound to the vector;
ii) Obtaining a fusion protein by expression of the vector of step i) in a host cell;
iii) Obtaining binding complex by binding of one or more biological regulators selected from the group consisting of fusion protein of step ii), protein, DNA/RNA, fat, carbohydrate and chemicals by chemical or physical covalent or non-covalent bond; and
iv) Mixed-culturing the binding complex of step iii) with cell cultures in vivo or ex vivo through routes including intramuscular, intraperitoneal, intravein, oral, nasal, subcutaneous, intradermal, mucosal or inhalation.

8. A method for delivering a biological regulator into eukaryotic or prokaryotic cytoplasm or nucleus, comprising steps:
i) Preparing peptide transducing recombinant expression vector which comprises a DNA encoding PTD, a DNA encoding one or more homologous or heterologous binding protein having DNA/RNA binding factor or DNA/RNA binding domain, and expression regulatory sequence operatively bound to the vector;
ii) Obtaining a fusion protein by expression of the vector of step i) in a host cell;
iii) Preparing a recombinant expression vector which comprises a DNA encoding a biological regulator, a NDA/RNA binding sequence specifically binding to the DNA/RNA binding factor or the DNA/RNA binding domain, and expression regulatory sequence bound operatively to the vector;
iv) Obtaining a binding complex by combining the fusion protein from step ii) with the recombinant expression vector from step iii); and
v) Mixed-culturing the binding complex of step iv) with cell cultures in vivo or ex vivo through routes including intramuscular, intraperitoneal, intravein, oral, nasal, subcutaneous, intradermal, mucosal or inhalation.

9. The method according to claim 7, wherein step ii) comprises an additional step combining NLS (Nuclear Localization Sequence) with PTD of fusion protein.

10. Protein transducing recombinant expression vector, wherein comprises a DNA encoding PTD, a DNA encoding encoding one or more homologous or heterologous binding protein, and expression regulatory sequence bound operatively to the vector.

11. The binding complex according to claim 2, wherein PTD is selected from the group consisting of Mph-1, Sim-2, Tat, R7, VP22, ANTP, MTS<Pep-1, and Pep-2.

12. The binding complex according to claim 2, wherein the biological regulator is a promoter or enhancer that expresses a gene specifically in specific species, tissues, organs or cells.

13. The binding complex according to claim 2, wherein the complex is delivered into cytoplasm or nucleus through routes including intramuscular, intraperitoneal, intravein, oral, nasal, subcutaneous, intradermal, mucosal or inhalation.

14. The method according to claim 8, wherein step ii) comprises an additional step combining NLS (Nuclear Localization Sequence) with PTD of fusion protein.


SEQUENCE LISTING (7 sequences)

SEQ ID NO: 1 - DNA, 75 bp, Artificial Sequence
  Designed nucleic acid sequence to act as a 5′ primer for preparing pSim2-Gal4
cgcggatccgccaaagccgcccgccaggccgcccggaagctactgtcttctatcgaacaagcatgcgatatttgc

SEQ ID NO: 2 - DNA, 81 bp, Artificial Sequence
  Designed nucleic acid sequence to act as a 5′ primer for preparing pMph1-Gal4
cgcggatcctatgcacgtgttcggaggcgtggaccccgccgcaagctactgtcttctatcgaacaagcatgcgatatttgc

SEQ ID NO: 3 - DNA, 84 bp, Artificial Sequence
  Designed nucleic acid sequence to act as a 5′ primer for preparing pTat-Gal4
cgcggatcctatggaaggaagaagaagcggagacaaagacgacgaaagctactgtcttctatcgaacaacgatgcgatatttgc

SEQ ID NO: 4 - DNA, 69 bp, Artificial Sequence
  Designed nucleic acid sequence to act as a 5′ primer for preparing pR7-Gal4
cgcggatccagaagaagaagaagaagaagaaagctactgtcttctatcgaacaacgatgcgatatttgc

SEQ ID NO: 5 - DNA, 21 bp, Artificial Sequence
  Designed nucleic acid sequence to act as a 3′ primer for preparing pSim2-Gal4, pMph1-Gal4, pR7-Gal4 and pTat-Gal4
ggaagatctcggcgatacagt

SEQ ID NO: 6 - DNA, 16 bp, Artificial Sequence
  Designed nucleic acid sequence to act as a 5′ primer for preparing Gal4 Binding Sequence (GBS)
aaggcctctcgaggac

SEQ ID NO: 7 - DNA, 16 bp, Artificial Sequence
  Designed nucleic acid sequence to act as a 3′ primer for preparing Gal4 Binding Sequence (GBS)
aaggccttttagcttc